(19) United States
(12) Patent Application Publication
(10) Pub. No.: US 2023/0414088 A1
(43) Pub. Date: Dec. 28, 2023

(54) METHODS AND SYSTEMS FOR AUTOMATED PNEUMATIC OTOSCOPY

(71) Applicant: OtoNexus Medical Technologies, Inc., Bellevue, WA (US)

(72) Inventors: Gina Kelly, Bothell, WA (US); Mark A. Moehring, Seattle, WA (US)

(73) Assignee: OtoNexus Medical Technologies, Inc., Bellevue, WA (US)

(21) Appl. No.: 18/358,762

(22) Filed: Jul. 25, 2023

Related U.S. Application Data
(63) Continuation of application No. PCT/US22/30605, filed on May 23, 2022.
(60) Provisional application No. 63/192,661, filed on May 25, 2021.

(51) Int. Cl. A61B 1/227; A61B 7/02; A61B 5/12; A61B 5/00

(52) U.S. Cl. CPC A61B 1/2275; A61B 7/023; A61B 5/126; A61B 5/7221

(57) ABSTRACT

An exemplary method for characterizing a quality of a membrane measurement may comprise receiving a reflected signal from a tympanic membrane in response to a pneumatic challenge; characterizing a quality of a seal in response to the reflected signal, wherein the quality of the seal is characterized based on a leak rate; and providing an indication that the leak rate is sufficiently small to continue with a measurement.

### CROSS-REFERENCE

This application is a continuation of PCT Application No. PCT/US22/30605, filed May 23, 2022, which claims priority to U.S. Provisional Patent Application No. 63/192,661 filed on May 25, 2021, which application is incorporated herein by reference in its entirety for all purposes.

### BACKGROUND

Acute otitis media (AOM) is an inflammatory process in the middle ear and is the most common clinical condition seen by pediatricians in children fifteen years and younger. AOM is generally associated with the presence of a middle ear effusion and is considered a middle ear inflammation. Complications of undiagnosed AOM can include hearing loss. Left untreated in children, recurrent AOM can also lead to delays in the development of speech and language skills.

### SUMMARY

Aspects of the present disclosure provide a method for characterizing a quality of a membrane measurement. The method may comprise: receiving a reflected signal from a tympanic membrane in response to a pneumatic challenge; characterizing a quality of a seal in response to the reflected signal, wherein the quality of the seal is characterized based on a leak rate; and providing an indication that the leak rate is sufficiently small to continue with a measurement.

In some embodiments, the method is implemented by a computer comprising a processor, wherein the characterizing and the providing are performed at the processor in response to the reflected signal.

In another aspect, the present disclosure provides a system for characterizing a quality of a membrane measurement. The system may comprise: a processor comprising instructions that when executed are configured to: process a reflected signal from a tympanic membrane in response to a pneumatic challenge; characterize a quality of a seal in response to the reflected signal, wherein the quality of the seal is characterized based on a leak rate; and provide an indication that the leak rate is sufficiently small to continue with a measurement.

In some embodiments, the processor is on board an otoscope. In some embodiments, the processor is on board a pneumatic otoscope.

In another aspect, the present disclosure provides a system comprising: a pressure source configured to provide a plurality of pressure profiles to a pneumatic volume comprising a target object or region; one or more sensors configured to detect or measure (i) a pressure within the pneumatic volume and/or (ii) a leak rate of the pneumatic volume; and a control unit configured to implement a closed loop control scheme to adjust or modulate an operation, a position, and/or a movement of the pressure source based on one or more measurements obtained using the one or more sensors.

In some embodiments, the system may further comprise a valve configured to equalize or a reset a pressure of the pneumatic volume. In some embodiments, the control unit may be configured to adjust an operation or a movement of the valve based on the one or more measurements obtained using the one or more sensors.

In some embodiments, the control unit may be configured to select or modify a pressure profile provided by the pressure source. In some embodiments, the control unit may be configured to select or modify the pressure profile based on an input provided by a user or an operator. In some embodiments, the input may comprise a selection of one or more operational modes. In some embodiments, the one or more operational modes may comprise a pressure scouting mode or a seal quality assessment mode. In some embodiments, the one or more operational modes may comprise a tympanic challenge mode or a tympanic response measurement mode. In some embodiments, the plurality of pressure profiles may comprise (i) a first pressure profile for a pressure scouting mode or a seal quality assessment mode and (ii) a second pressure profile for a tympanic challenge mode or a tympanic response measurement mode. In some embodiments, the first pressure profile and the second pressure profile may be different.

In some embodiments, the control unit may comprise a pressure monitor configured to override an operation or a movement of a release valve for an air manifold in pneumatic communication with the pressure source, based on the one or more measurements. In some embodiments, the pressure monitor may be configured to control or modulate an operation or a movement of the pressure source based on the one or more measurements. In some embodiments, the control unit and/or the pressure monitor may be configured to determine a seal quality for the pneumatic volume based on (i) the one or more measurements or (ii) an amount of movement or displacement needed for the pressure source to achieve or maintain a threshold pressure for the pneumatic volume.

In some embodiments, the system may further comprise an indicator for providing an indication of the seal quality to a user or an operator. In some embodiments, the indication may comprise an auditory, visual, or haptic alert or feedback.

In some embodiments, the pressure source comprises an electroacoustic device. In some embodiments, the electroacoustic device comprises a speaker. In some embodiments, the electroacoustic device comprises an air impulse generator or air pump configured to displace a volume of air in the pneumatic volume.

In some embodiments, the pneumatic volume comprises a sealed or partially sealed volume or region between the pressure source and the target object or region. In some embodiments, the pneumatic volume extends from the pressure source to at least an ear canal of a patient or a subject.

In some embodiments, the system may further comprise one or more additional sensors configured to detect (i) one or more signals received, transmitted, or reflected from the target object or region, and/or (ii) a behavior or a movement of the target object or region in response to one or more of the plurality of pressure profiles. In some embodiments, the one or more additional sensors may comprise a microphone.

In some embodiments, the target object or region may comprise a biological membrane. In some embodiments, the biological membrane may comprise a tympanic membrane.

In another aspect, the present disclosure provides a method, comprising: (a) using a pressure source to provide a plurality of pressure profiles to a pneumatic volume comprising a target object or region; (b) using one or more sensors to detect or measure (i) a pressure within the pneumatic volume and/or (ii) a leak rate of the pneumatic volume; and (c) using a control unit configured to implement a closed loop control scheme to adjust or modulate an operation, a position, and/or a movement of the pressure source based on one or more measurements obtained using the one or more sensors.

In some embodiments, the method may further comprise, subsequent to (c), measuring a behavior or a movement of the target object or region based at least in part on one or more signals received, transmitted, or reflected from the target object or region in response to one or more of the plurality of pressure profiles. In some embodiments, (c) may further comprise using a valve to equalize or a reset a pressure of the pneumatic volume. In some embodiments, the method may further comprise adjusting an operation or a movement of the valve based on the one or more measurements obtained using the one or more sensors.

In some embodiments, the method may further comprise, subsequent to (a), selecting or modifying a pressure profile to be provided by the pressure source. In some embodiments, the plurality of pressure profiles may correspond to one or more operational modes. In some embodiments, the one or more operational modes may comprise a pressure scouting mode or a seal quality assessment mode. In some embodiments, the one or more operational modes may comprise a tympanic challenge mode or a tympanic response measurement mode. In some embodiments, the plurality of pressure profiles may comprise (i) a first pressure profile for a pressure scouting mode or a seal quality assessment mode and (ii) a second pressure profile for a tympanic challenge mode or a tympanic response measurement mode. In some embodiments, the first pressure profile and the second pressure profile may be different.

In some embodiments, the method may further comprise overriding an operation or a movement of a release valve for an air manifold in pneumatic communication with the pressure source, based on the one or more measurements.

In some embodiments, the method may further comprise determining a seal quality for the pneumatic volume based on (i) the one or more measurements or (ii) an amount of movement or displacement needed for the pressure source to achieve or maintain a threshold pressure for the pneumatic volume. In some embodiments, the method may further comprise providing an indication of the seal quality to a user or an operator. In some embodiments, the indication may comprise an auditory, visual, or haptic alert or feedback.

In some embodiments, the pneumatic volume may comprise a sealed or partially sealed volume or region between the pressure source and the target object or region. In some embodiments, the pneumatic volume may extend from the pressure source to at least an ear canal of a patient or a subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows an exemplary system for characterizing a membrane, in accordance with some embodiments.

FIG. 1B shows an example of a pneumatic tympanic challenge system (TCS), in accordance with some embodiments.

FIG. 1C shows an example of a TCS comprising a pressure monitoring system, in accordance with some embodiments.

FIG. 2A illustrates an example of a pressure transient profile for evaluating TM behavior or response, in accordance with some embodiments.

FIG. 2B illustrates an example of a pressure transient profile for performing a pressure scout function, in accordance with some embodiments.

FIG. 3 illustrates an exemplary configuration for an ear bud style external ear canal seal, in accordance with some embodiments.

FIG. 4A illustrates exemplary user interfaces indicating the quality of a seal, in accordance with some embodiments.

FIG. 4B illustrates additional exemplary user interfaces indicating the quality of a seal, in accordance with some embodiments.

FIG. 5 schematically illustrates a computer system that is configured to implement methods of the present disclosure.

### DETAILED DESCRIPTION

In system with a manual insufflation bulb, the trained practitioner (clinician) elicits a sharp pressure transient, both negative and positive pressure on a lightly sealed external ear canal, in order to observe the dampened motion of the tympanic membrane (TM) in a patient with suspected otitis media (OM). A healthy middle ear (no OM and no effusion) will exhibit a “snappy” motion in response to the pressure transients. With middle ear effusion, the TM loses some of its snappy response.

The clinician may use an external elastomeric bulb-style seal about 5 mm distal to the open end of the speculum in order to seal to the external ear canal. Any leak in the system may be overcome by the clinician squeezing harder on the bulb and can generate as much as 520 mm H2O to 748 mm H2O of pressure on the TM. See, for example, Clarke L, Wiederhold M, Gates G. Quantitation of pneumatic otoscopy. Otolaryngol Head Neck Surg. February 1987; 96(2):119-24. PMID: 3120084, and Cavanaugh R. Pediatricians and the pneumatic otoscope: are we playing it by ear? Pediatrics. August 1989; 84(2):362-4. PMID: 2748268, each of which is incorporated by reference herein in their entireties. This pressure approaches the threshold for patient injury. The differential pressure across the TM required for rupture varies between 17 to 100 kPa. See, for example, Cameron J, Skofronick J, Grant R. Physics of the Body (Medical Physics Series). Madison WI: Medical Physics Publishing; 1999, and Richmond D, Yelverton J, Phillips Y, Fletcher E. Physical correlates of eardrum rupture. Ann Otol Rhinol Laryngol Suppl. May 1989; 140:35-41, each of which is incorporated by reference herein in their entireties. Assuming the worst-case differential pressure from the literature, 17 kPa is assumed to be the lower threshold for TM rupture which is approximately 1,734 mm H2O.

Systems and methods of the present disclosure may improve upon these methods by automating the process of adjusting for a leak in a pneumatic otoscopy measurement. Systems and methods described herein may impart a highly repeatable, low pressure transient on the TM and measuring TM motion. Systems and methods disclose herein may be used in conjunction with a variety of sensors including without limitation: air-coupled ultrasound, coherent optical or infrared light, or incoherent optical or infrared light.

### Pneumatic Tympanic Challenge System (TCS)

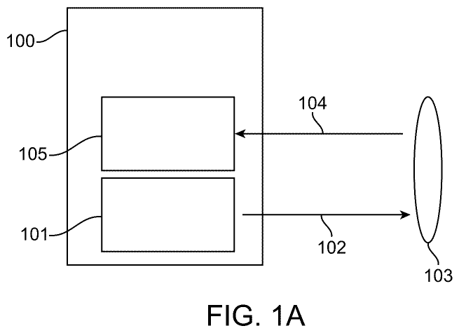

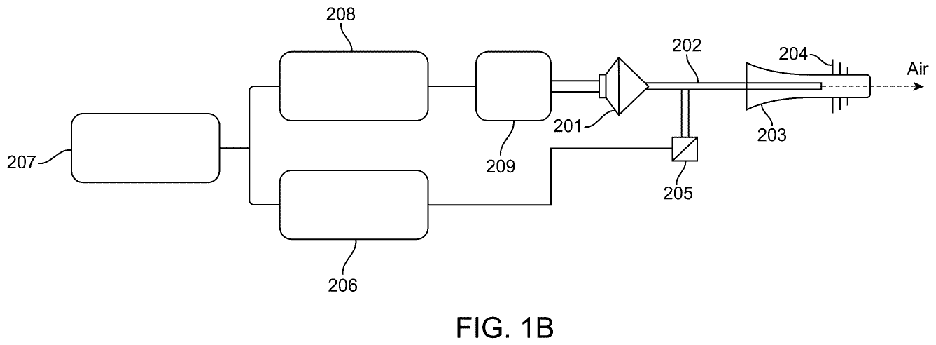

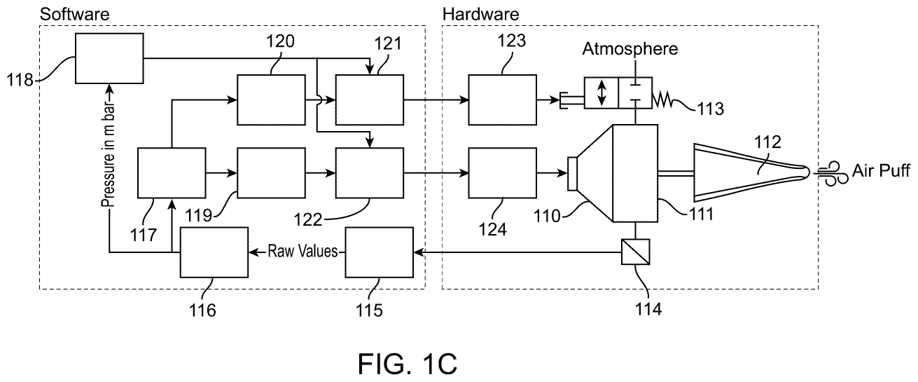

FIG. 1A shows an exemplary system 100 for characterizing a response of a membrane. The system 100 may comprise an electroacoustic device 101 configured to generate a signal 102 that is transmitted to a target region 103. The signal 102 may comprise a pressure transient profile. The target region 103 may comprise, for example, a tympanic membrane (TM).

In some cases, the pressure transient profile may contain two or more cycles of negative, neutral and positive pressure components. The cycles in both pressure directions may allow for measurement of the degree of symmetry or asymmetry in TM motion in response to the pressure transient profile.

In some cases, the signal 102 may interact with the target region 103 and induce a movement of the target region 103. The signal 102 may be reflected back towards the system 100 as a reflected signal 104. The reflected signal 104 may be produced by and/or received from the tympanic membrane in response to a pneumatic challenge involving or initiated by the interaction between the pressure transient profile associated with the signal 102 and the target region 103.

In some cases, the reflected signal 104 may be received at a detector 105. The detector 105 may comprise, for example, a pressure sensor or a microphone. The detector 105 may be configured to detect and/or characterize a response or a movement of the membrane after the interaction between the signal 102 and the membrane. In some cases, the detector 105 may be configured to determine and/or characterize a quality of a seal that is formed between a speculum and the external ear canal of a patient or a subject. The speculum may be provided as part of the system 100, or may be a separate, add-on accessory that is couplable or attachable to the system 100 or any portion or component thereof.

FIG. 1B shows an example of a pneumatic tympanic challenge system (TCS), in accordance with some embodiments. A pneumatic TCS may comprise an electroacoustic device, e.g., an audio speaker 201, which acts as a positive displacement air impulse generator (air pump) that can be finely tuned to adjust the volume or pressure of a pneumatic volume comprising a target object or region (e.g., a TM membrane). In some cases, the pneumatic volume may comprise a contiguous pneumatic volume comprising at least the speaker 201, an internal volume of any manifolds, conduits, channels, or pneumatic piping 202 provided between the speaker 201 and a speculum 203, an inside volume of the speculum 203, and an external ear canal of a subject or a patient. The speculum 203 may be used to create an external ear canal seal 204. In some cases, the pneumatic volume may comprise a sealed or partially sealed volume or region between a pressure source (e.g., the electroacoustic device) and a target object or region (e.g., the TM membrane). In some cases, the pneumatic volume may extend from the pressure source to at least an ear canal of a patient or a subject.

In some embodiments, the position, direction of travel, rate of travel, pressure profile, and/or total displacement of the TCS (e.g., audio speaker) may be adjusted manually or automatically (e.g., using a closed loop control as described elsewhere herein). The speaker may be controlled by an analog driving voltage which may in turn be controlled by digital/analog conversion devices 208 controlled by a microprocessor 207.

In some cases, the TCS system may operate in a closed-loop mode which uses one or more pressure sensors 205 to measure pressure and/or leak rate within the pneumatic volume and to adjust an operation or a configuration of the TCS system according to the measured pressure or leak rate. The one or more pressure sensors 205 may be operatively coupled to a proportional integrative differential controller 206 and/or a microprocessor 207 that is configured to adjust an operation of the TCS or one or more components or subsystems of the TCS. The pressure measurements obtained using the one or more pressure sensors may be used to control the motion of the audio speaker through a proportional integrative differential control (PID) scheme in order to maintain a required or desired pressure profile. In some cases, the proportional integrative differential controller 206 and/or the microprocessor 207 may be configured to control an operation of an audio driver 209 that is configured to move or displace the speaker 201.

FIG. 1C schematically illustrates a block diagram of an exemplary tympanic challenge system (TCS). The system may comprise an electroacoustic device 110 (e.g., an audio speaker) that is configured to operate as a positive displacement air impulse generator (air pump) that is connected and/or sealed to an air manifold 111.

In some embodiments, the system can be finely tuned to adjust the contiguous pneumatic volume of the speaker 110, the air manifold 111, the piping between the air manifold and a speculum 112, an inside portion or region of the speculum 112, and an external ear canal of a subject or a patient. In some cases, the position, direction of travel, rate of travel, pressure profile, and/or total displacement of the electroacoustic device 110 may be adjusted manually or automatically (e.g., using a closed loop control).

In some embodiments, the system may comprise an equalization valve 113. The equalization valve 113 may be connected to or integrated with the air manifold 111. The equalization valve 113 may be configured to regulate the pressure in the contiguous pneumatic volume and to allow the contiguous pneumatic volume to be set back to a desired pressure or to atmospheric pressure (i.e., zero gage pressure) at the end of a fully positive or fully negative displacement limit of the electroacoustic device 110 (e.g., when the electroacoustic device 110 is maximally displaced in a particular direction towards or away from a center position or a starting location of the electroacoustic device 110). The equalization valve 113 can be configured to reset or recover the pressure capacity of the electroacoustic device 110.

In some embodiments, the TCS system may be configured to operate in a closed loop pressure mode. The closed loop pressure mode may utilize feedback from a pressure sensor 114. The pressure sensor 114 may be configured to measure pressure which in turn feeds signals indicating the measured pressure through a serial engine sample processing unit 115, a pressure interpretation module 116, and a proportional integrative differential (PID) control 117 that is configured to output one or more control signals based on the feedback received from the pressure sensor 114.

In some cases, the pressure interpretation module 116 may be configured to process raw data values received from the serial engine sample processing unit 115 to compute pressure values (e.g., pressure in mbar) that are usable by a pressure monitor subsystem 118 to control an operation of the valve 113 and/or the electroacoustic device 110. In some cases, the pressure monitor subsystem 118 may be used to control or implement a valve state override 121 or a speaker cutoff 122.

In some embodiments, the pressure monitor subsystem 118 may be a separable software block operating outside of the pressure control logic associated with the PID control 117. The pressure monitor subsystem 118 may be configured to examine the measured pressure for approach to any safety limits (e.g., proof or burst limits), and to (i) override the valve state to open and/or (ii) cutoff any further speaker motion until the limits have been cleared.

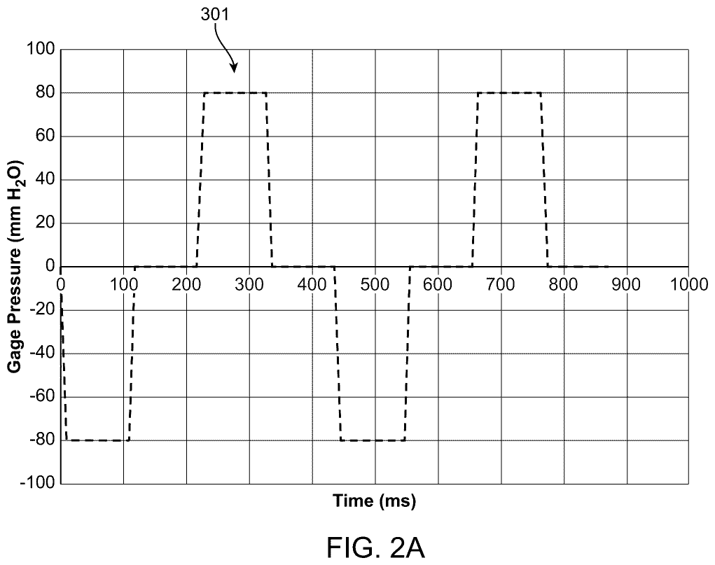

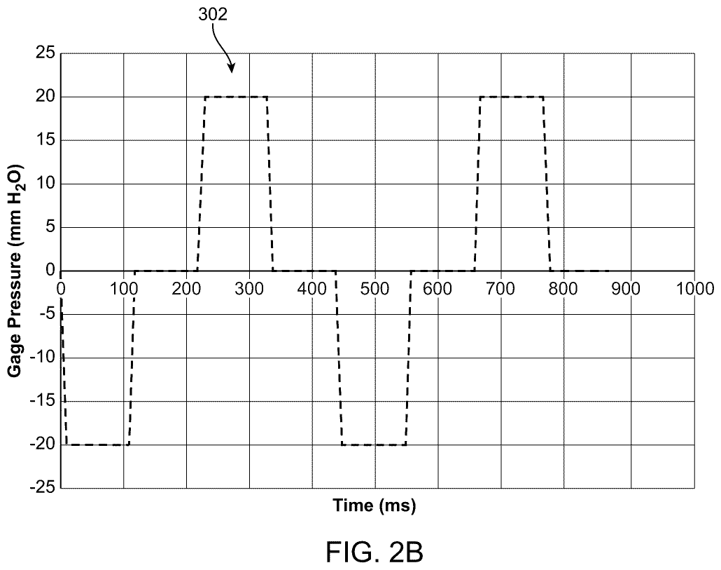

In some embodiments, the PID control 117 may reside in a common software block with a pressure control logic that can configure the system or the electroacoustic device/speaker/pressure source to generate a pre-programmed time-varying square wave pressure profile (e.g., as shown in FIG. 2A or FIG. 2B). The pressure control logic and the PID control signal can be provided as inputs for both speaker control 119 and valve control 120. The speaker control 119 and the valve control 120 can be used to modulate an operation of the valve 113 and/or an operation or a movement of the electroacoustic device 110 (e.g., using a valve driver 123 or a motor controller 124).

FIG. 2A illustrates an example pressure transient profile 301 in accordance with some embodiments. The pressure transient profile may comprise two or more cycles of negative, neutral and positive pressure components. The cycles in both pressure direction may allow for measurement of the degree of symmetry or asymmetry in TM motion, which may reveal aspects of effusion behind the TM inside the middle ear. In some cases, the pressure transient profile may comprise a pressure profile that cycles at ±80 mm H₂O. In some cases, a different pressure transient profile may be used for pressure scouting purposes, as described in further detail below.

For measurements of TM mobility, the pre-programmed pressure transient profile imparted on the TM may comprise a plurality of cycles of negative, neutral and positive pressure components. The plurality of cycles of negative, neutral and positive pressure components can interact with the TM to induce a response or behavior (e.g., TM motion). In some cases, the induced response or behavior may exhibit a degree of symmetry or asymmetry that can indicate a presence or an absence of effusion behind the TM inside the middle ear of a patient or a subject.

The pressure transient profile may allow for observation of drag (dampening) in movement of the TM which may be caused by effusion behind the TM. A healthy TM with no effusion in the middle ear will exhibit a TM displacement which exhibits a profile with a high degree of similarity to the pressure profile. By contrast, a middle ear with effusion may exhibit a dampened profile making it possible to diagnose the disease state and type of effusion inside the middle ear.

Systems and methods of the present disclosure may overcome at least some limitations in conventional systems and methods for pneumatic otoscopy. For instance, the systems and methods of the present disclosure may provide a consistent pneumatic pressure profile that is applied to the TM regardless of patient ear canal volume, volumetric leak (subject to the operation of the pressure scout as described in greater detail below), and operator technique. The consistent pressure profile may allow patient-to-patient baselining for middle ear effusion state. The systems and methods of the present disclosure may also provide a greater margin to a safety limit on the TM because there is no need to over pressurize the patient ear canal volume in order to observe TM movement. Further, the presently disclosed systems and methods do not require an operator to squeeze an insufflation bulb to apply positive and negative air pressure to the TM, thereby allowing the operator to make more efficient use of time with the patient, especially very young pediatric patients who may not provide long periods of compliance for pneumatic otoscopy.

FIG. 2B illustrates an example pressure transient profile 302 that can be used for pressure scouting purposes. In some embodiments, the TCS or the electroacoustic device or audio speaker may be operated in a pressure scout mode and/or may be configured to implement a pressure scout function. In some cases, the pressure scout function may move or operate the electroacoustic device or audio speaker using a pressure profile that is different than the pressure profile used to stimulate or an interrogate the TM. For example, instead of using a pressure profile of ±80 mm H₂O, the system may cycle at ±20 mm H₂O in order to perform pressure scouting. The pressure scout function may allow a constant monitoring of pressure, leak rate, and/or pressure change in response to the motion or movement of the electroacoustic device or audio speaker while maintaining a pressure profile that is very tolerable or even barely sensed by the patient. In some cases, the TCS can predict how well the detected or estimated leak rate would perform during a measurement cycle that utilizes, for example, a pressure profile comprising a ±80 mmH₂O measurement cycle.

In some cases, the TCS may be configured to use a first pressure transient profile for measuring TM movement or response. In some cases, the TCS may be configured to use a second pressure transient profile for pressure scouting. The second pressure transient profile may comprise a maximum or minimum value having an absolute value that is less than an absolute value of a maximum or minimum value of the first pressure transient profile.

In some cases, the first pressure transient profile and/or the second pressure transient profile may be preset, pre-programmed, or otherwise predetermined. In other cases, the first pressure transient profile and/or the second pressure transient profile may be adjustable (e.g., based on patient need or operator preference). In some cases, the first pressure transient profile and/or the second pressure transient profile may be automatically or autonomously adjusted by the TCS using a control loop as described elsewhere herein.

### Seal

Systems and methods of the present disclosure may be integrated and/or used compatibly with a wide range of speculum seal designs. In some non-limiting embodiments, a speculum seal comprising multiple flanges of elastomeric material may be used to form a seal between the TCS system and the external ear canal of a subject or a patient. A speculum seal with multiple flanges may conform to the variability in shapes of the external ear canal, provide a torturous path for air to escape from/to the external ear canal, and lower the contact force on the highly innervated portions of the external ear canal, thus reducing any patient discomfort. The seal may be designed based on a body of pediatric radiographic data of ear canals from the helix to the tympanic membrane.

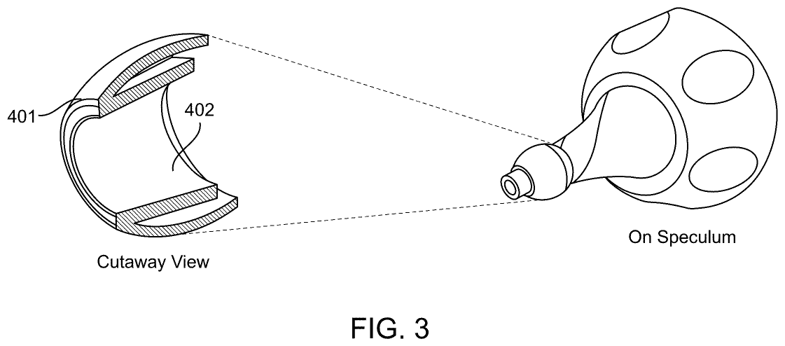

In some alternative embodiments, an ear bud style external ear canal seal may be used to form a seal between the TCS system and the external ear canal of a subject or a patient. FIG. 3 illustrates an exemplary configuration for an ear bud style external ear canal seal. In some cases, the seal may be installed over or on a tip of a speculum. As shown in the cutaway view, the ear bud style external ear canal seal may comprise a plurality of surfaces comprising an outer surface 401 and an inner surface 402. The inner surface 402 may seal to the outside of the speculum shell, and the outer surface 401 may seal to the skin in the external ear canal of the subject or patient. In some cases, the two sealing surfaces 401, 402 can articulate independently of each other while still staying connected. In some cases, the ear bud style external ear canal seal may comprise an elastomer (e.g., a low durometer elastomer). The elastomer may provide a relatively high coefficient of friction to grip and seal on both the speculum shell and the skin in the external ear canal of the subject or patient.

In some cases, the outer surface 401 and the inner surface 402 may be at least partially joined. For example, the outer surface 401 and the inner surface 402 may be joined at one or more locations. The one or more locations may correspond to an intersection between a region or a portion of the outer surface 401 and a region or a portion of the inner surface 402. In some cases, the seal may comprise a gap, a cavity, a recess, or an internal volume between the outer surface 401 and the inner surface 402. The gap, cavity, recess, or internal volume may span between the outer surface 401 and the inner surface 402. The perimeter or boundary of the gap, cavity, recess, or internal volume may coincide with the one or more locations at which the outer surface 401 and the inner surface 402 are joined. The gap, cavity, recess, or internal volume may allow the outer surface 401 and the inner surface 402 to articulate independently of each other while still staying connected.

The ear bud style external ear canal seal design may be used in combination with the closed loop pressure control schemes described elsewhere herein to enable accurate monitoring of pressure in situ and precise control and/or modulation of the electroacoustic device or speaker depending on the sensed pressure. As described elsewhere herein, the control and/or modulation of the electroacoustic device or speaker may comprise controlling or modulating a position, direction of travel, rate of travel, pressure profile, and/or total displacement of the electroacoustic device or speaker.

The ear bud style external ear canal seal design may also be used in combination with a pressure scout function or mode as described in greater detail below. The pressure scout may aid an operator in assessing a seal quality and establishing or re-establishing an acceptable seal on the external ear canal of a patient or a subject.

### Pressure Scout

When using a TCS or a pressure profile to assess a characteristic or a response of a TM, there may be instances where the operator may not obtain an adequate seal or may not be aware of an excessive leak until they attempt to take a TM mobility measurement with the TCS. If the leak is significant, the pressure PID control may respond by moving, displacing, or otherwise modulating an operation of the electroacoustic device or audio speaker to make up for the lost volume of air between the TM and the speculum. In some embodiments, e.g., in the extreme case of an excessive leak, the electroacoustic device or audio speaker may reach its physical displacement limit (even with equalization valve action) before a full TCS measurement cycle can be completed. In such cases, the operator may not know of the leak until the warning of the TCS voice coil has timed out. The systems and methods of the present disclosure may provide a proactive pressure indication to the operator, referred to herein as a pressure scout function or mode.

In some embodiments, the TCS or the electroacoustic device or audio speaker may be operated in a pressure scout mode and/or may be configured to implement a pressure scout function. In some cases, the pressure scout function may move or operate the electroacoustic device or audio speaker using a pressure profile that is different than the pressure profile used to stimulate or interrogate the TM. For example, instead of using a pressure profile of ±80 mm H₂O, the system may cycle at ±20 mm H₂O in order to perform pressure scouting. The pressure scout function may allow a constant monitoring of pressure, leak rate, and/or pressure change in response to the motion or movement of the electroacoustic device or audio speaker while maintaining a pressure profile that is tolerable or even barely sensed by the patient or subject. The software and/or processing modules of the TCS may predict how well the external ear canal is sealed based on how much additional speaker movement is required. For instance, if very little movement is required, there is a good seal. Above a certain threshold of a “good seal” (which threshold may correspond to, for example, an amount of movement required for the electroacoustic device or audio speaker in order to achieve a desired leak rate or pressure), the TCS can predict how well the detected or estimated leak rate would perform during a measurement cycle that utilizes, for example, a pressure profile comprising a ±80 mmH₂O measurement cycle. In some cases, the system may also predict or determine whether the electroacoustic device or audio speaker may have ample margin to effect the measurement cycle without nearing the limits of audio speaker displacement. In cases where the system does predict or determine that the electroacoustic device or audio speaker may have ample margin to effect a partial or full measurement cycle without nearing the limits of audio speaker displacement, the system may output an indication to the operator that an acceptable seal has been formed. Conversely, if there is too much of an air leak beyond the abilities or capacity of the TCS to maintain a desired pressure for a particular pneumatic volume, the operator may be presented with an indication of an unacceptable seal.

In any of the embodiments described herein, the indication may comprise an auditory, a visual, or a tactile indication. In some cases, the indication for an acceptable seal may be different than an indication for an unacceptable seal.

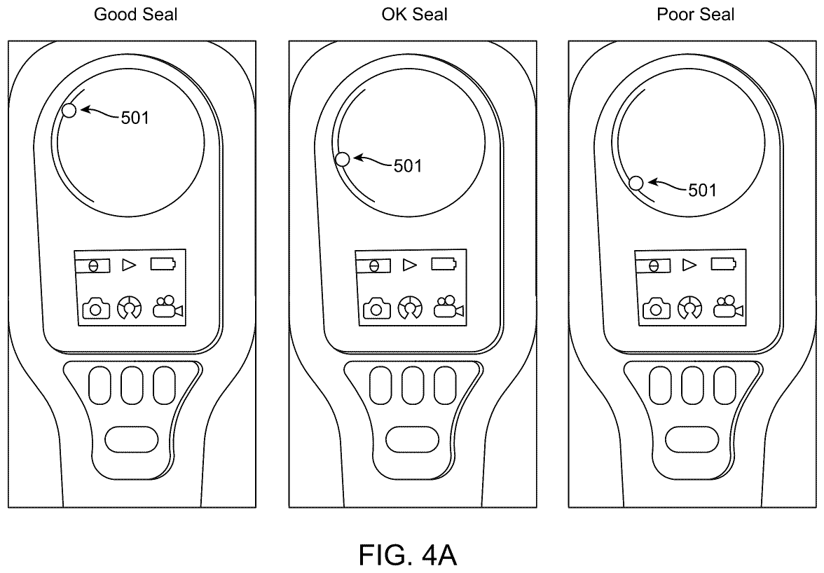

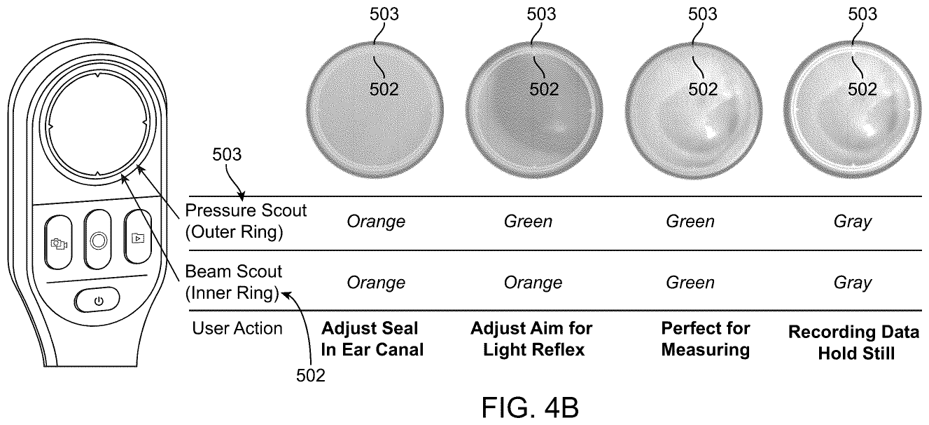

FIG. 4A illustrates exemplary user interfaces indicating the quality of a seal, in accordance with some embodiments. Information regarding a quality of the seal may be presented in the form of sliding ball 501 which can move up and down on an arced scale. When the ball 501 is high on the scale, it may turn green to indicate to the operator that the external ear canal leak conditions are acceptable for taking measurements. If the ball 501 is lower on the scale with a yellow color, there may just be an adequate seal, and the pressure scout function may prompt the operator to make one or more adjustments to obtain a desired pneumatic pressure or leak rate for the pneumatic volume. In some cases, if the ball 501 is positioned towards a lower region of the scale and colored red, the pressure scout function may indicate that a leak rate is too excessive for a measurement to be accurately taken. In this way, the system may proactively, in situ, inform the operator about the pneumatic seal conditions.

FIG. 4B illustrates additional exemplary user interfaces indicating the quality of a seal, in accordance with some embodiments. The quality of the seal may be presented in the form of two concentric rings superimposed on the periphery of an otologic display.

In some cases, the two concentric rings may comprise an inner ring 502 and an outer ring 503. The outer ring 503 may correspond to the pressure scout indication. The inner ring 502 may correspond to a beam scout indication. The beam scout indication may be based on ultrasound returns and/or the signal or wave properties of the ultrasound returns, and can indicate whether a portion of the TM is moving in phase or out of phase with the TCS pressure waveform. The movement of the TM in phase with the TCS pressure waveform may indicate that the TM is being interrogated as opposed to another nearby tissue region such as skin on the inside of a patient's ear canal wall. In some cases, the movement of the TM out of phase with the TCS pressure waveform may indicate that another nearby tissue region is being interrogated as opposed to the TM.

In some cases, when the inner ring 502 and the outer ring 503 are displayed in a first color (e.g., orange), the conditions required for TM mobility measurements may not be satisfied. In such cases, the TCS may prompt the user to adjust a seal formed for an ear canal of a patient or a subject. In some cases, when the inner ring 502 is displayed in a first color (e.g., orange) and the outer ring 503 is displayed in a second color (e.g., green), the conditions required for TM mobility measurements may not be satisfied, and the TCS may prompt the user to adjust his or her aim for light reflex. In some non-limiting embodiments, when the outer ring 503 is displayed in a first color (e.g., orange) and the inner ring 502 is displayed in a second color (e.g., green), the conditions required for TM mobility measurements may not be satisfied (e.g., because a leak rate may be excessive even if the TM is in ultrasonic view), in which case the TCS may prompt the user to reposition the speculum tip inside the patient's ear canal to obtain a better seal. In some cases, if both rings 502, 503 are displayed in a second color (e.g., green), the rings may indicate that the conditions for measuring TM mobility have been satisfied. In some cases, if both rings 502, 503 are displayed in a third color (e.g., gray), the TCS may prompt the user to hold still so that the TCS can begin or continue recording data. In some embodiments, a software control may be implemented such that the beam scout (inner ring) does not turn green until the pressure scout (outer ring) has already turned green. This can encourage operators to establish an acceptable seal first since the repositioning of the speculum tip to achieve an acceptable seal may or may not place the TM within ultrasonic view of the TCS. Once the speculum tip is positioned to achieve the acceptable seal, the software control may allow the TCS to evaluate whether the TM is moving in phase with the TCS pressure waveform, and whether the TM is in ultrasonic view (which can then indicate to the operator that they may proceed with pneumatic otoscopy).

In some embodiments, when the TCS is started for clinical operation outside of a subject's ear, both the inner ring 502 and the outer ring 503 may be displayed on a display screen in orange. As a user (e.g., a physician or a clinician) navigates the speculum, along with the integrated external ear canal seal, into the ear canal and forms a satisfactory seal, the pressure scout (outer ring 503) may turn green. When the light reflex is brought into view and the ultrasound Doppler indicates that the TM motion is in phase with the pressure perturbations, the beam scout (inner ring 502) may turn green. When both rings 502, 503 are colored green, the TCS may automatically and/or autonomously begin taking and recording pressure measurements and/or measurements for TM mobility. As the system processes and records measurement data (e.g., using the control systems or closed loop controls described above and referenced in FIG. 1B and/or FIG. 1C), the rings 502, 503 may turn gray as an indication to the operator to hold the system in place.

This pressure scout may overcome various limitations with existing systems and methods for pneumatic otoscopy. For example, the normal hand tremor of the operator which is nominally about 8 to 23 Hz can affect the position of the speculum tip inside the ear canal and cause the pneumatic seal, pneumatic pressure, and/or leak rate to fluctuate. The pressure scout function may detect when such tremors result in a pneumatic seal, pneumatic pressure, and/or leak rate that is unacceptable or insufficient for measuring TM response or behavior, so that an operator can take corrective action.

The presently disclosed systems and method may provide numerous advantages over existing systems and methods for pneumatic otoscopy. For example, not all ear canals have the same volume, but the systems and methods of the present disclosure may utilize a closed loop pressure control along with a pressure scout function to allow an operator to adjust or improve a seal between the speculum and the external ear canal in order to accommodate different size ear canals. The presently disclosed systems and methods may provide real-time, easy to interpret feedback to an operator preparing for or performing pneumatic otoscopy, thereby allowing the operator to use his or her time more efficiently with younger non-compliant pediatric patients who may not be inclined to comply with the operator's instructions or requests for extended periods of time.

The TCS systems disclosed herein may be of sufficient volume and may provide sufficient air pressure inside the tip of the speculum (e.g., at the narrowest point of the tip of the speculum), such that the corresponding Reynold's number in that volume remains well below the transition boundary from laminar to turbulent flow (Re<1,000), which ensures that turbulent air flow does not interfere with the air-coupled ultrasound sensing methods disclosed herein.

### Computer Systems

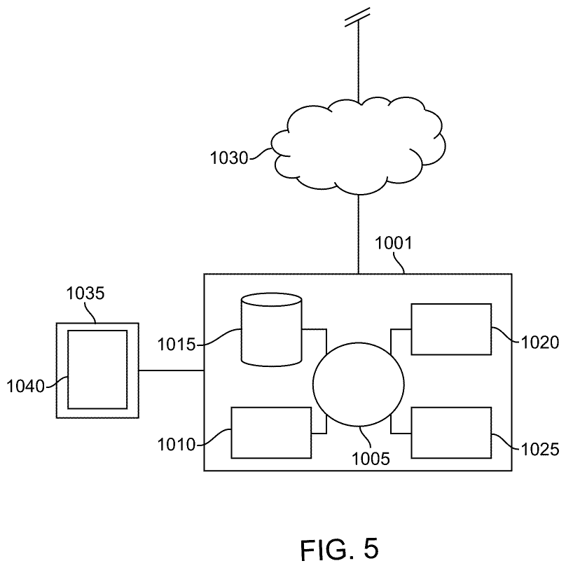

In an aspect, the present disclosure provides computer systems that are programmed or otherwise configured to implement methods of the disclosure, e.g., any of the subject methods for pneumatic otoscopy. FIG. 5 shows a computer system 1001 that is programmed or otherwise configured to implement a method for pneumatic otoscopy. The computer system 1001 may be configured to, for example, control, adjust, or modulate an operation of (i) a valve driver for resetting a pneumatic pressure of a pneumatic volume and/or (ii) a motor controller for moving or displacing an electroacoustic device or speaker. The pneumatic volume may comprise the electroacoustic device or speaker, an air manifold, piping between the air manifold and a speculum, an inside portion or region of the speculum, and a sealed external ear canal of a patient or a subject. The computer system 1001 can be an electronic device of a user or a computer system that is remotely located with respect to the electronic device. The electronic device can be a mobile electronic device.

The computer system 1001 may include a central processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet. The network 1030 in some cases is a telecommunication and/or data network. The network 1030 can include one or more computer servers, which can enable distributed computing, such as cloud computing. The network 1030, in some cases with the aid of the computer system 1001, can implement a peer-to-peer network, which may enable devices coupled to the computer system 1001 to behave as a client or a server.

The CPU 1005 can execute a sequence of machine-readable instructions, which can be embodied in a program or software. The instructions may be stored in a memory location, such as the memory 1010. The instructions can be directed to the CPU 1005, which can subsequently program or otherwise configure the CPU 1005 to implement methods of the present disclosure. Examples of operations performed by the CPU 1005 can include fetch, decode, execute, and writeback.

The CPU 1005 can be part of a circuit, such as an integrated circuit. One or more other components of the system 1001 can be included in the circuit. In some cases, the circuit is an application specific integrated circuit (ASIC).

The storage unit 1015 can store files, such as drivers, libraries and saved programs. The storage unit 1015 can store user data, e.g., user preferences and user programs. The computer system 1001 in some cases can include one or more additional data storage units that are located external to the computer system 1001 (e.g., on a remote server that is in communication with the computer system 1001 through an intranet or the Internet).

The computer system 1001 can communicate with one or more remote computer systems through the network 1030. For instance, the computer system 1001 can communicate with a remote computer system of a user (e.g., a doctor, a clinician, a physician, a medical worker or assistant, a healthcare provider, an imaging technician, etc.). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1001 via the network 1030.

Methods as described herein can be implemented by way of machine (e.g., computer processor) executable code stored on an electronic storage location of the computer system 1001, such as, for example, on the memory 1010 or electronic storage unit 1015. The machine executable or machine readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media including, for example, optical or magnetic disks, or any storage devices in any computer(s) or the like, may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 can include or be in communication with an electronic display 1035 that comprises a user interface (UI) 1040 for providing, for example, a portal for a healthcare provider or an imaging technician to monitor or track a seal quality for a target pneumatic volume of interest and/or various measurements such as pressure scout measurements and/or TM response measurements. The portal may be provided through an application programming interface (API). A user or entity can also interact with various elements in the portal via the UI. Examples of UI's include, without limitation, a graphical user interface (GUI) and web-based user interface.

Methods and systems of the present disclosure can be implemented by way of one or more pressure control logic algorithms. A pressure control logic algorithm can be implemented by way of software upon execution by the central processing unit 1005. For example, the pressure control logic algorithm may be configured to adjust an operation of a valve or an electroacoustic device or speaker based on one or more pressure measurements obtained for a target pneumatic volume of interest.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for characterizing a quality of a membrane measurement, the method comprising:
receiving a reflected signal from a tympanic membrane in response to a pneumatic challenge;
characterizing a quality of a seal in response to the reflected signal, wherein the quality of the seal is characterized based on a leak rate; and
providing an indication that the leak rate is sufficiently small to continue with a measurement.

2. A system for characterizing a quality of a membrane measurement, the system comprising:
a processor comprising instructions that when executed are configured to:
receive a reflected signal from a tympanic membrane in response to a pneumatic challenge;
characterize a quality of a seal in response to the reflected signal, wherein the quality of the seal is characterized based on a leak rate; and
provide an indication that the leak rate is sufficiently small to continue with a measurement.

3. A system for automated pneumatic otoscopy, comprising:
a pressure source configured to provide a plurality of pressure profiles to a pneumatic volume comprising a target object or region;
one or more sensors configured to detect or measure (i) a pressure within the pneumatic volume and/or (ii) a leak rate of the pneumatic volume; and
a control unit configured to implement a closed loop control scheme to adjust or modulate an operation, a position, and/or a movement of the pressure source based on one or more measurements obtained using the one or more sensors.

4. The system of claim 3, further comprising a valve configured to equalize or a reset a pressure of the pneumatic volume.

5. The system of claim 4, wherein the control unit is configured to adjust an operation or a movement of the valve based on the one or more measurements obtained using the one or more sensors.

6. The system of claim 3, wherein the control unit is configured to select or modify a pressure profile provided by the pressure source.

7. The system of claim 6, wherein the control unit is configured to select or modify the pressure profile based on an input provided by a user or an operator.

8. The system of claim 7, wherein the input comprises a selection of one or more operational modes.

9. The system of claim 8, wherein the one or more operational modes comprise a pressure scouting mode or a seal quality assessment mode.

10. The system of claim 8, wherein the one or more operational modes comprise a tympanic challenge mode or a tympanic response measurement mode.

11. The system of claim 3, wherein the plurality of pressure profiles comprise (i) a first pressure profile for a pressure scouting mode or a seal quality assessment mode and (ii) a second pressure profile for a tympanic challenge mode or a tympanic response measurement mode.

12. The system of claim 11, wherein the first pressure profile and the second pressure profile are different.

13. The system of claim 3, wherein the control unit comprises a pressure monitor configured to override an operation or a movement of a release valve for an air manifold in pneumatic communication with the pressure source, based on the one or more measurements.

14. The system of claim 13, wherein the pressure monitor is configured to control or modulate an operation or a movement of the pressure source based on the one or more measurements.

15. The system of claim 14, wherein the control unit and/or the pressure monitor is configured to determine a seal quality for the pneumatic volume based on (i) the one or more measurements or (ii) an amount of movement or displacement needed for the pressure source to achieve or maintain a threshold pressure for the pneumatic volume.

16. The system of claim 15, further comprising an indicator for providing an indication of the seal quality to a user or an operator.

17. The system of claim 16, wherein the indication comprises an auditory, visual, or haptic alert or feedback.

18. The system of claim 3, wherein the pressure source comprises an electroacoustic device.

19. The system of claim 18, wherein the electroacoustic device comprises a speaker.

20. The system of claim 18, wherein the electroacoustic device comprises an air impulse generator or air pump configured to displace a volume of air in the pneumatic volume.

21. The system of claim 3, wherein the pneumatic volume comprises a sealed or partially sealed volume or region between the pressure source and the target object or region.

22. The system of claim 3, wherein the pneumatic volume extends from the pressure source to at least an ear canal of a patient or a subject.

23. The system of claim 3, further comprising one or more additional sensors configured to detect (i) one or more signals received, transmitted, or reflected from the target object or region, and/or (ii) a behavior or a movement of the target object or region in response to one or more of the plurality of pressure profiles.

24. The system of claim 23, wherein the one or more additional sensors comprise a microphone.

25. The system of claim 3, wherein the target object or region comprises a biological membrane.

26. The system of claim 25, wherein the biological membrane comprises a tympanic membrane.

27. A method for automated pneumatic otoscopy, comprising:
(a) using a pressure source to provide a plurality of pressure profiles to a pneumatic volume comprising a target object or region;
(b) using one or more sensors to detect or measure (i) a pressure within the pneumatic volume and/or (ii) a leak rate of the pneumatic volume; and
(c) using a control unit configured to implement a closed loop control scheme to adjust or modulate an operation, a position, and/or a movement of the pressure source based on one or more measurements obtained using the one or more sensors.
